(19) United States
(12) Patent Application Publication
(10) Pub. No.: US 2021/0161092 A1
(43) Pub. Date: Jun. 3, 2021

(54) SECONDARY METABOLITE SCREENING SYSTEM

(71) Applicants: Ajikumar Parayil KUMARAN, Cambridge, MA (US); Abdul Hakkim RAHAMATHULLAH, BOSTON, MA (US); Deborah MCEWAN, BOSTON, MA (US); Frederick AUSUBEL, BOSTON, MA (US)

(72) Inventors: Ajikumar Parayil KUMARAN, Cambridge, MA (US); Abdul Hakkim RAHAMATHULLAH, BOSTON, MA (US); Deborah MCEWAN, BOSTON, MA (US); Frederick AUSUBEL, BOSTON, MA (US)

(21) Appl. No.: 15/998,665

(22) PCT Filed: Feb. 16, 2017
(86) PCT No.: PCT/US2017/018060

§ 371 Date: Aug. 16, 2018

Related U.S. Application Data
(60) Provisional application No. 62/295,834, filed on Feb. 16, 2016.

(51) Int. Cl. A01H 3/04; C12Q 1/02; G01N 33/50

(52) U.S. Cl. CPC A01H 3/04; G01N 33/5097; G01N 33/5082; C12Q 1/025

(57) ABSTRACT

The present invention relates to systems and methods for screening natural products such as secondary metabolites produced by engineered microbial strains.

### PRIORITY

This application claims the benefit of, and claims priority to, U.S. Provisional Application No. 62/295,834 filed Feb. 16, 2016, which is hereby incorporated by reference in its entirety.

### FIELD OF THE INVENTION

The present invention relates to systems and methods for screening natural product scaffolds (e.g., secondary metabolites), which in some embodiments are produced by combinatorial metabolic engineering of microbial strains.

### BACKGROUND OF THE INVENTION

Natural products offer a rich reservoir of chemically diverse bioactive molecules with therapeutic potentials. For example, of all new small molecule drugs approved by the United States Food and Drug Administration (FDA) between 1981 and 2014, over 65% are either natural products or natural product derivatives. These numbers are even higher for certain therapeutic areas, including cancers (83%) and infectious diseases (78%).

Nevertheless, for several reasons natural products are significantly underrepresented in small molecule libraries for drug delivery. For example, natural products are often produced at extremely low levels by their native organisms, and most high-throughput screens do not function well with small amounts of low purity material. Further, there is a bias against natural products in the pharmaceutical industry because they are either difficult to synthesize, or it is not practical to purify sufficient quantities of a particular natural product from the native organism, or the natural product cannot be readily or reliably sourced.

Accordingly, there remains a need for high throughput systems and methods for screening compounds based upon natural product scaffolds for bioactivity coupled with systems and methods for producing the natural product scaffold in high yield.

### SUMMARY OF THE INVENTION

The present invention relates to methods and systems for screening candidate agents for bioactivity by providing a cell or organism exhibiting a measurable phenotype, and contacting the cell or organism with a library of microbial strains created by combinatorial metabolic engineering, or contacting the cell or organism with a material derived from a culture of the microbial strains. The microbial strains produce a library of compounds based on a selected natural product scaffold. Active agents that affect the measurable phenotype can be identified from the library, and further characterized from the corresponding microbial strain, including identification and characterization of the corresponding biosynthetic pathway. In some embodiments, strains producing bioactive compounds are identified, and optimized for production of the bioactive agent in large quantities by fermentation.

In various embodiments, the present invention provides a library of microbial strains that produces thousands of discrete secondary metabolites based upon a desired natural compound scaffold through combinatorial overexpression of heterologous biosynthetic enzymes. Exemplary natural product scaffolds include, but are not limited to, terpenes, terpenoids, alkaloids, cannabinoids, steroids, saponins, glycosides, stilbenoids, polyphenols, flavonoids, antibiotics, polyketides, fatty acids, or non-ribosomal peptides. In various embodiments, the microbial strain is a bacterium, optionally selected from an E. coli, Pseudomonas spp., Enterococcus spp., Bacillus spp., and Staphylococcus spp. In other embodiments, the microbial strain is an archaea, a fungus, or yeast.

For example, the microbial strain can be engineered to produce a terpenoid library through combinatorial overexpression of biosynthetic enzymes. In some embodiments, the strain library is a bacterial strain engineered to overexpress one or more MEP pathway enzymes and a prenyltransferase enzyme to produce a desired core substrate, along with combinatorial expression of terpene synthase (TPS) enzymes to thereby create a terpene or terpenoid library. The strain library may further express, including in combinatorial fashion, cytochrome P450 enzymes and/or P450 reductase partners and/or other enzymes for decorating the terpene scaffold with chemical groups including hydroxyl, ketone, alkyl (e.g., methyl), acetyl, aldehyde, glycosyl, aryl (e.g., benzyl), among others.

In various embodiments, the microbial strains (producing a library of secondary metabolites) or material derived from cultures of the microbial strains, are screened by adding to cells or small organisms contained within wells of a multiwell plate. In some embodiments, the cell or organism is a plant cell, a plant, or a plant part. In other embodiments, the cell is of a small vertebrate organism or is an embryo. In yet other embodiments, the cell or organism is a fungi or yeast. Additionally, the organism may be a protozoan, a cnidarian, a flatworm, an arthropod, an amoeba, a paramecium, or a nematode. In some embodiments, the organism is the nematode Caenorhabditis elegans (C. elegans), which also provides the advantage of being bacterivorous. In some embodiments, the cell or organism is engineered to express a human gene, which may be a pharmaceutical target or a marker for toxicity.

In various embodiments, the method involves plating the cell or organism in multiwell plates, contacting individual wells with at least one strain from the library (or a material derived therefrom), and screening the cell or organism for a measurable phenotype. In various embodiments, the screening is in high throughput. In some embodiments, the measurable phenotype to be assessed is induction or reduction of gene expression or protein expression, protein modification, metabolism, change in metabolic or physiologic state, subcellular or tissue structure and organization, protein or RNA stability, epigenetic modification, cell or organism death, lifespan extension, autophagy, organellar structure and function, intracellular or intercellular trafficking or signaling, neuronal functioning, cell proliferation, RNA toxicity, a stress response, a pathogen response, calcium influx, fat storage, developmental timing, brood size, or behavior such as social feeding or food avoidance.

In another aspect, the present invention provides the use of a library of engineered microbial strains for in vitro screening assays. In such embodiments, the microbial strains or material derived therefrom may be plated in multiwell plates and contacted with in vitro assay targets, which may be whole organisms engineered to exhibit a phenotype of interest, or in some embodiments, the assay targets are isolated molecular targets of interest.

In various embodiments, the methods identify active agents that are subsequently formulated as a fungicide or pesticide. In some embodiments, the methods identify active agents that are subsequently formulated for application to plants. In some embodiments, the identified active agents are formulated as an insecticide or repellant. In yet further embodiments, the methods identify agents that are subsequently formulated as pharmaceutical compositions for the prevention or treatment of various human or animal conditions or diseases.

Additional aspects and embodiments of the invention will be apparent from the following detailed disclosure.

### DESCRIPTION OF THE FIGURES

FIG. 1 provides an exemplary illustration of the multivariate modular metabolic engineering (MMME) platform and high-throughput compound screening platform of certain embodiments.

### DETAILED DESCRIPTION OF THE INVENTION

The present invention relates to systems and methods for screening compound libraries based on natural products, the compound libraries being produced by metabolic engineering of microbial strains. Natural products provide a rich reservoir of compounds, including novel compounds, for identifying bioactivities, including for drug discovery and pest control among other things. However, tapping into this rich resource remains challenging as these natural products are typically secondary metabolites produced at extremely low levels by their native organisms. This further poses significant hurdles for high throughput screening which conventionally requires a high quantity and quality synthesis of the testing substance. The present invention overcomes these barriers by synthesizing a library of secondary metabolites (e.g., based on a selected natural compound scaffold) through combinatorial metabolic engineering, and in some embodiments provides convenient screening of this library (e.g., without compound isolation) in a whole-cell or whole-organism screening platform.

In one aspect, the present invention provides a microbial platform for producing a library of secondary metabolites based on a selected natural product scaffold. Particularly, synthetic biology and metabolic engineering approaches are provided to reconstruct biosynthetic pathways in microbial strains in a combinatorial fashion.

Secondary metabolites are organic compounds that typically are not directly involved in the normal growth, development or reproduction of organisms. Instead, secondary metabolites are often involved in defenses against predators, parasites and diseases, or are required for interspecies competition or to facilitate the reproductive processes. In various embodiments, the secondary metabolites are a class naturally present in plant, fungal, or bacterial sources. Exemplary secondary metabolites include, but are not limited to, terpene, terpenoid, alkaloid, cannabinoid, steroid, saponin, glycoside, stilbenoid, polyphenol, flavonoid, antibiotic, polyketide, fatty acid, non-ribosomal peptide, or ribosomal peptide. In various embodiments, core enzymes for scaffold synthesis are overexpressed, together with combinatorial expression of one or more subsequent enzymatic steps, and/or with varying expression levels, with the potential of synthesizing a large variety of compounds. For example, in some embodiments, the microbial strains overexpress a library of enzymes potentially capable of polymerizing or cyclizing the scaffold, and/or removing or decorating the scaffold with various functional groups. Various pathways are known for producing core scaffolds based on secondary metabolites present in plant and fungal species, for example.

In various embodiments, the microbial cells express one or more heterologous genes so as to produce a core scaffold for structural diversification in one or more subsequent enzymatic steps. It should be appreciated that some cells may express an endogenous copy of one or more of the genes involved in scaffold synthesis, as well as one or more heterologous copies to complement the natural expression levels. In some embodiments, the core substrate for structural diversification is a prenyl diphosphate compound.

In an exemplary embodiment, the microbial cell is engineered to produce a terpene or terpenoid library. As used herein, “terpene” refers to a large and varied class of hydrocarbons that have a simple unifying feature, despite their structural diversity. According to the “isoprene rule”, all terpenes include isoprene (C5) units. This fact is used for a rational classification depending on the number of such units. Monoterpenes comprise 2 isoprene units and are classified as (C10) terpenes, sesquiterpenes comprise 3 isoprene units and are classified as (C15) terpenes, diterpenes comprise 4 isoprene units and are classified as (C20) terpenes, sesterterpenes (C25), triterpenes (C30). They occur as acyclic or mono- to pentacyclic derivatives with alcohol, ether, ester, aldehyde, or ketone groups (the so called “terpenoids”). Terpenes such as Monoterpenes (C10), Sesquiterpenes (C15) and Diterpenes (C20) are derived from the prenyl diphosphate substrates, geranyl diphosphate (GPP), farnesyl diphosphate (FPP) and geranylgeranyl diphosphate (GGPP) respectively through the action of a very large group of enzymes called the terpene (terpenoid) synthases (TPS). These enzymes are often referred to as terpene cyclases since the product of the reactions are cyclised to various monoterpene, sesquiterpene and diterpene carbon skeleton products. Many of the resulting carbon skeletons undergo subsequence oxygenation by cytochrome P450 oxidase enzymes (P450 enzymes) to give rise to large families of derivatives. In exemplary embodiments, the microbial cells are engineered to produce a monoterpene or monoterpenoid library, a sesquiterpene or sesquiterpenoid library, or diterpene or diterpenoid library, or a triterpene or triterpenoid library.

In various embodiments, the microbial cell overexpresses one or more genes involved in the terpene or terpenoid pathway, as disclosed, for example, in WO 2011/060057, U.S. Pat. Nos. 8,927,241, and 8,512,988, the entire disclosures of all of which are hereby incorporated by reference.

For example, the microbial cell may be engineered to overexpress one or more genes involved in the MEP pathway for terpene synthesis. The MEP (2-C-methyl-D-erythritol 4-phosphate) pathway, also called the MEP/DOXP (2-C-methyl-D-erythritol 4-phosphate/1-deoxy-D-xylulose 5-phosphate) pathway or the non-mevalonate pathway or the mevalonic acid-independent pathway refers to the pathway that converts glyceraldehyde 3-phosphate and pyruvate to IPP and DMAPP. The pathway typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA. In some embodiments, the microbial cell is engineered to have at least one or more additional copies of an MEP pathway gene, such as dxs, ispD, ispF, and/or idi gene (e.g., dxs and idi; or dxs, ispD, ispF, and/or idi).

In some embodiments, the microbial cell is engineered to express one or more genes involved in the mevalonate (MVA) pathway for terpene synthesis. The MVA pathway refers to the biosynthetic pathway that converts acetyl-CoA to IPP. The mevalonate pathway typically comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-CoA) (e.g., by action of HMG-CoA synthase (HMGS)); (c) converting HMG-CoA to mevalonate (e.g., by action of HMG-CoA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)).

In exemplary embodiments, the microbial strain may overexpress a prenyltransferase enzyme, such as a geranyl diphosphate synthase (GPS), a geranylgeranyl diphosphate synthase (GGPS), a farnesyl diphosphate synthase (FPS), or a farnesyl geranyl diphosphate synthase (FGPPS). The resulting microbial strain thus produces a core compound (“a prenyl diphosphate compound”), optionally selected from geranyl diphosphate, a geranylgeranyl diphosphate, farnesyl diphosphate, and farnesyl geranyl diphosphate, which can act as a substrate for a library of terpene synthase enzymes.

In various embodiments, the microbial strain is engineered to express in a combinatorial fashion a library of terpene synthase enzymes, which can be monoterpene synthase enzymes, sesquiterpene synthase enzymes, diterpene synthase enzymes, sesterterpene synthase enzymes, triterpene synthase enzymes etc., depending on the selected prenyl diphosphate compound substrate. Exemplary terpene synthase enzymes include enzymes selected from plant or fungal sources, which are publically available and/or which can be identified in additional species through bioinformatics analysis. Exemplary fungal sources for terpene synthases include species of Basiodiomycota and Ascomycota. Exemplary synthases are described, for example, in U.S. Pat. No. 8,927,241, which is hereby incorporated by reference in its entirety. In some embodiments, the microbial library expresses at least about 100 terpene synthase enzymes, or at least about 500 terpene synthase enzymes, or at least about 1000 terpene synthase enzymes, or at least about 2000 terpene synthase enzymes.

In some embodiments, terpenoid synthases are generated in part through modification of wild type or parent terpene synthase enzymes. For example, one or more amino acids in the active site can be substituted (including in a combinatorial fashion) to create new functionalities within a parent enzyme. Structural coordinates common to terpene synthases, including active site coordinates, are disclosed in U.S. Pat. No. 6,645,762, which is hereby incorporated by reference in its entirety. In some embodiments, a library of terpene synthase enzymes is created from parent enzymes by combinatorial substitution of from 2 to 10 amino acids, or from 4 to 10 amino acids. In some embodiments, at least one substitution is of an amino acid in the terpene synthase active site.

In some embodiments, the library of microbial strains further varies the expression level of core metabolic enzymes (e.g., MEP or MVA pathway enzymes) with respect to one or more synthetic enzymes introduced to generate compound diversity (e.g., terpene synthase). For example, strains may vary expression levels of heterologous enzymes by varying promoter strength, ribosome binding site, expression of genes together in modules (e.g., operons), and/or gene copy number. By varying relative enzyme expression levels, amounts and ratios of terpene and terpenoid products can be diversified in the library.

Manipulation of the expression of genes and/or proteins, including gene modules, can be achieved through various methods. For example, expression of genes or operons can be regulated through selection of promoters, such as inducible or constitutive promoters, with different strengths (e.g., strong, intermediate, or weak). Several non-limiting examples of bacterial promoters of different strengths include Trc, T5 and T7. Additionally, expression of genes or operons can be regulated through manipulation of the copy number of the gene or operon in the cell. In some embodiments, expression of genes or operons can be regulated through manipulating the order of the genes within a module, where the genes transcribed first are generally expressed at a higher level. In some embodiments, expression of genes or operons is regulated through integration of one or more genes or operons into the chromosome.

Gene expression can also be varied through selection of promoters and modification of ribosomal binding sites, as well as in some embodiments, selection of high-copy number plasmids, or single-, low- or medium-copy number plasmids.

Expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. Cells are genetically engineered by the introduction into the cells of heterologous DNA. The heterologous DNA is placed under operable control of transcriptional elements to permit the expression of the heterologous DNA in the host cell.

The strain library may further express, optionally in combinatorial fashion, cytochrome P450 oxidase enzymes and/or P450 reductase partners and/or other enzymes for altering or decorating the terpene scaffold with chemical groups including hydroxyl, ketone, alkyl (e.g., methyl), acetyl, aldehyde, glycosyl, aryl (e.g., benzyl), among others.

In various embodiments, the microbial library expresses a panel or library of P450 oxidase enzymes. P450 enzymes are important oxidizing enzymes involved in the metabolic pathways of thousands of natural products. Table 1 below provides a list of exemplary P450 enzymes. P450 enzymes (including P450 reductase counterparts) can be identified from various plant or fungal sources. An exemplary non-limiting list of P450 enzymes includes those of Table 1. P450 oxidase enzymes can be expressed as fusion proteins with a corresponding P450 reductase, in some embodiments.

TABLE 1

| Species | Name | Native Substrate | Native Reaction Product |
|---|---|---|---|
| Zingiber | zzHO | α-humulene | 8-hydroxy-α-humulene |
| zerumbet | | | |
| Barnadesia | BsGAO | germacrene A | germacra-1(10),4,11(13)- |
| spinosa | | | trien-12-ol |
| Hyoscyamus | HmPO | premnaspirodiene | solavetivol |
| muticus | | | |
| Latuca | LsGAO | germacrene A | germacra-1(10),4,11(13)- |
| spicata | | | trien-12-ol |
| Nicotiana | NtEAO | 5-epi-aristolochene | capsidiol |
| tabacum | | | |
| Citrus × | CpVO | valencene | nootkatol |
| paradisi | | | |
| Artemesia | AaAO | amorphadiene | artemisinic acid |
| annua | | | |
| Arabidopsis | AtKO | kaurene | kaurenoic acid |
| thaliana | | | |
| Stevia | SrKO | kaurene | kaurenoic acid |
| rebaudiana | | | |
| Pseudomonas | PpKO | kaurene | kaurenoic acid |
| putida | | | |
| Bacillus | BM3 | fatty acids | hydroxylated FAs |
| megaterium | | | |
| Cichorium | CiVO | valencene | nootkatone |
| intybus | | | |
| Helianthus | HaGAO | germacrene A | germacrene A acid |
| annuus | | | |

In certain embodiments, P450 enzymes are selected from plant or fungal sources, which are publically available and/or which can be identified in additional species through bioinformatics analysis. Exemplary fungal sources include species of Basiodiomycota and Ascomycota.

In certain embodiments, the N-terminus of the P450 enzymes may be modified to increase their functional expression in bacterial host cells, as described, for example, in WO2016/029153, the entire disclosure is hereby incorporated by reference.

In some embodiments, P450s are generated in part through modification of wild type or parent enzymes. For example, one or more amino acids can be substituted (including in a combinatorial fashion) to create new functionalities within a single parent enzyme. In some embodiments, a library of P450 oxidase enzymes is created from parent enzymes by combinatorial substitution of from 5 to 20 amino acids, or from 5 to 10 amino acids. In some embodiments, at least one substitution is of an amino acid in an active site or putative active site.

In some embodiments, the library expresses at least about 100 discrete P450 oxidase enzymes, or at least about 500 P450 oxidase enzymes, or at least about 1000 P450 oxidase enzymes, or at least about 2000 P450 oxidase enzymes.

In various embodiments, the microbial strains express a library of uridine diphosphate dependent glycosyltransferase enzymes (UGT), methyltransferase enzymes, acetyltransferase enzymes, and/or benzoyl transferase enzymes. In an exemplary embodiment, the microbial cell is engineered to express a UGT enzyme as described, for example, in WO2016/073740, the entire disclosure is hereby incorporated by reference. Such enzymes may likewise be diversified by mutation, to provide further compound structure diversity. In some embodiments, the library expresses at least about 100 discrete enzymes in accordance with this paragraph, or at least about 500 enzymes, or at least about 1000 enzymes, or at least about 2000 enzymes in accordance with this paragraph.

In specific embodiments, expression of the one or more heterologous genes are regulated in a modular fashion (i.e., multiple genes are regulated together as a module) so as to increase production of a secondary metabolite. By way of example, the genes involved in terpene production may be regulated as an upstream (MEP) pathway module (e.g., containing one or more genes of the MEP pathway) and a downstream pathway module as described in WO 2011/060057, U.S. Pat. Nos. 8,927,241, and 8,512,988, the entire disclosures are hereby incorporated by reference. Upstream and downstream modules may be expressed under control of promoters with different strengths (e.g., in combinatorial fashion), to further diversify the terpene and terpenoid products.

In various embodiments, the microbial library is based on any prokaryotic or eukaryotic organism that can be engineered to express one or more heterologous genes. In some embodiments, the microbial cell is a bacterial cell, such as, but not limited to, Escherichia spp., Enterococcus spp., Bacillus spp., or Staphylococcus spp., Streptomyces spp., Zymomonas spp., Acetobacter spp., Citrobacter spp., Synechocystis spp., Rhodobacter spp., Rhizobium spp., Clostridium spp., Corynebacterium spp., Streptococcus spp., Xanthomonas spp., Lactobacillus spp., Lactococcus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Therms spp., Stenotrophomonas spp., Chromobacterium spp., Sinorhizobium spp., Saccharopolyspora spp., Agrobacterium spp., and Pantoea spp. In an embodiment, the bacterial cell is a Gram-positive cell such as a species of Bacillus. In another embodiment, the bacterial cell is a Gram-negative cell such as an Escherichia coli (E. coli). In some embodiments, the microbial cell is selected from E. coli, Bacillus subtilis, or Pseudomonas putida.

In some embodiments, the microbial cell is an archaea. In exemplary embodiments, the archaea may include, but is not limited to, Aeropyrum spp., Cenarchaeum spp., Haladaptatus spp., Haloarcula spp., Halobacterium spp., Halobiforma spp., Haloferax spp., Haloquadratum spp., Halorubrum spp., Metallosphaera spp., Methanobrevibacter spp., Methanocella spp., Methanococcoides spp., Methanogenium spp., Methanosarcina spp., Methanosphaera spp., Methanothrix spp., Methylosphaera spp., Nanoarchaeum spp., Palaeococcus spp., Picrophilus spp., Pyrococcus spp., Pyrodictium spp., Pyrolobus spp., Sulfolobus spp., and Thermococcus spp.

In some embodiments, the microbial cell is a fungal cell such as a yeast cell. Exemplary fungal cells include, but are not limited to, Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp., and industrial polyploid yeast strains. Other examples of fungal cells include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In an embodiment, the microbial cell is a yeast, and may be a species of Saccharomyces, Pichia, Schizosaccharomyces, or Yarrowia, including Saccharomyces cerevisiae, Pichia pastoris, Schizosaccharomyces pombe, and Yarrowia lipolytica.

Microbial strains may be added to the screening assay (described below) as whole cells or extracts, or alternatively, material from cultures are separated and used for screens. For example, cell culture products can be recovered for screening, optionally without extraction and purification. In some embodiments, recovery can include partitioning the desired product into an organic phase or hydrophobic phase. Alternatively, the aqueous phase can be recovered, or the whole cell biomass can be recovered for screening, optionally with some processing to remove cellular debris. The production and characterization of products can be determined and/or quantified, for example, by gas chromatography (e.g., GC-MS).

In some embodiments, the secondary metabolite is a metabolite that partitions into an organic or hydrophobic phase, such terpene and/or terpenoid. For example, terpene and/or terpenoid product oil is extracted from aqueous reaction medium using an organic solvent, such as an alkane such as heptane or dodecane. In other embodiments, product oil is extracted from aqueous reaction medium using a hydrophobic phase, such as a vegetable oil. Vegetable oil containing terpene and/or terpenoid products is a convenient material for screening, and the vegetable oil can be tolerated by many organisms for whole-cell screening.

The present invention provides methods for screening active agents by contacting the engineered microbial cells that produce a secondary metabolite, or a material derived from the microbial cells or culture, with a cell or organism exhibiting a measurable phenotype (“a recipient cell or organism”). The recipient cell or organism is suitable for screening in multiwell plates. In various embodiments, the organism is selected from a fungus, a protist, a hydrozoa, a planaria, a nematode, an insect, a plant or plant part, or a microbe.

In some embodiments, the organism is a protozoan such as an amoeba, flagellate, ciliate, or sporozoan. In an embodiment, the protozoan is Tetrahymena thermophila. In some embodiments, the organism is an amoeba or paramecium. In some embodiments, the protozoan is Dictyostelium discoideum.

In some embodiments, the organism is an aquatic organism such as a cnidarian. Exemplary cnidaria include, but are not limited to, Hydra vulgaris and Nematostella vectensis.

In some embodiments, the organism is a flatworm such as a flatworm belonging to the phylum Platyhelminthes. In an exemplary embodiment, the flatworm is Schmidtea mediterranea. In some embodiments, the organism is a roundworm such as a nematode. In an exemplary embodiment, the nematode is Caenorhabditis elegans (C. elegans).

In some embodiments, the organism is an arthropod. Exemplary arthropods that may be used include, but are not limited to, Drosophila melanogaster, Anopheles aegypti, or Anopheles gambiae.

In some embodiments, the recipient organism or cell is a plant cell, a plant, or a plant part. Exemplary plants include, but are not limited to, a higher plant; dicotyledonous plant; monocotyledonous plant; consumable plant (e.g., crop plants and plants used for their oils); soybean; rapeseed; linseed; corn; safflowers; sunflowers; tobacco; a plant of the family Fabaceae (Leguminosae, legume family, pea family, bean family, or pulse family); or a plant of the genus Glycine; peanut; Phaseolus vulgaris, Vicia faba; Pisum sativum; and Arabidopsis thaliana. In some embodiments, the selected plants are derived from members of the taxonomic family known as the Gramineae. This includes all members of the grass family of which the edible varieties are known as cereals. The cereals include a wide variety of species such as wheat (Triticum sps.), rice (Oryza sps.) barley (Hordeum sps.) oats, (Avena sps.) rye (Secale sps.), corn (maize) (Zea sps.) and millet (Pennisettum sps.). In an embodiment, the organism is Arabidopsis thaliana.

In some embodiments, the plant, plant cell, or plant part is of a bryophyte. Bryophyte refers to all embryophytes (land plants) that are non-vascular plants, such as mosses, hornworts, and liverworts. In still other embodiments, the plant, plant cell, or plant part is a fern.

In various embodiments, the recipient organism or cell is an algal cell, for example, a green alga, a red alga, or a brown alga. In certain embodiments, the alga is a microalga, for example and without limitation, a Chlamydamonas ssp., Dunaliella ssp., Haematococcus spp., Scenendesmus spp., Chlorella spp. or Nannochloropsis spp. More particular examples, include, without limitation, Chlamydomonas reinhardtii, Dunaliella saline, Haematococcus pluvialis, Scenedesmus dimorphus., D. viridis, and D. tertiolecta. Examples of organisms contemplated for use herein include, but are not limited to, rhodophyta, chlorophyta, heterokontophyta, tribophyta, glaucophyta, chlorarachniophytes, euglenoids, haptophyta, cryptomonads, dinoflagellata, and phytoplankton.

In some embodiments, the cell is a cell of a non-human vertebrate organism or an embryo. In an embodiment, the organism for use in screening is a zebrafish such as Danio rerio.

In some embodiments, the recipient organism or cell is a fungi such as a yeast. Any of the fungi or yeast cells described herein for microbial engineering (e.g. Saccharomyces spp., Schizosaccharomyces spp., Pichia spp., Paffia spp., Kluyveromyces spp., Candida spp.) may also be used for screening. Exemplary fungi or yeast cell includes, but is not limited to, pathogenic organism such as Candida albicans, Botrytis cineria, Aspergillus fumigatus, Aspergillus nidulans, Fusarium oxysporum Cryptococcus spp., such as C. neoformans, C. laurentii, and C. kuetzingi.

In some embodiments, the cell is a mammalian cell or cell line. Exemplary mammalian cells or cell lines includes, but are not limited to, primary mammalian cells, or cell lines such as COS-1 or COS-7 (monkey kidney-derived), L-929 (murine fibroblast-derived), C127 (murine mammary tumor-derived), 3T3 (murine fibroblast-derived), CHO (Chinese hamster ovary-derived; including DHFR CHO), HeLa (human cervical cancer-derived), BHK (hamster kidney fibroblast-derived, e.g., BHK21), PER.C6 (human embryonic retinal cells), HEK-293 (human embryonic kidney-derived), VERO-76 (African green monkey kidney cells), HELA (human cervical carcinoma cells), MDCK (canine kidney cells), BRL 3A (buffalo rat liver cells), W138 (human lung cells), Hep G2 (human liver cells), HKB11 cells (a somatic cell fusion between human kidney and human B cells), MMT 060562 (mouse mammary tumor cells), TRI cells, MRC 5 cells, FRhL-2 cells, Jurkat, FS4 cells, and myeloma cells (e.g., Y0, NS0, Sp2/0, NS1, Ag8, and P3U1).

In various embodiments, the recipient cell or organism exhibits a measurable phenotype, and the present methods comprise identifying whether a secondary metabolite in the library affects the measurable phenotype. In various embodiments, the measurable phenotype includes, but is not limited to, induction or reduction of gene expression or protein expression, protein modification, metabolism, change in metabolic or physiologic state, subcellular or tissue structure and organization, protein or RNA stability, epigenetic modification, cell or organism death, lifespan extension, autophagy, organellar structure and function, intracellular or intercellular trafficking or signaling, neuronal functioning, cell proliferation, RNA toxicity, a stress response, a pathogen response, calcium influx, fat storage, developmental timing, brood size, or behavior such as social feeding or food avoidance.

In various embodiments, the measurable phenotype is determined by one or more of, assaying pathogen response, stress response, detoxification, hypoxia response, unfolded protein response, mitochondrial marker(s), RNAi function, piRNA function, microRNA function, and/or proteasome function.

In some embodiments, the measurable phenotype is the activity of a subcellular or intercellular signaling pathway such as, but not limited to, the Wnt pathway, P38 MAP kinase pathway, bZIP pathway, insulin/IGF pathway, G-protein coupled receptor pathway, RTK-Ras-MAPK pathway, Tor pathway, and/or TGF-b pathway.

In some embodiments, the effect on said measurable phenotype is quantified by the level of protein expression of a reporter gene and/or cellular location of the reporter gene RNA or protein, or impact on morphology or motility. In some embodiments, the cell or organism is engineered to express a gene (e.g., a human gene), which is optionally a pharmaceutical target or marker for toxicity.

In various embodiments, measurable phenotype is quantified by the protein expression of a reporter gene, which may be increased or decreased in response to the presence of a candidate molecule in the library.

In some embodiments, the reporter gene encodes a luminescent or fluorescent protein. Exemplary luminescent or fluorescent proteins include, for example, luciferase, a modified luciferase protein, blue/UV fluorescent proteins (for example, TagBFP, Azurite, EBFP2, mKalama1, Sirius, Sapphire, and T-Sapphire), cyan fluorescent proteins (for example, ECFP, Cerulean, SCFP3A, mTurquoise, monomeric Midoriishi-Cyan, TagCFP, and mTFP1), green fluorescent proteins (for example, EGFP, Emerald, Superfolder GFP, Monomeric Azami Green, TagGFP2, mUKG, and mWasabi), yellow fluorescent proteins (for example, EYFP, Citrine, Venus, SYFP2, and TagYFP), orange fluorescent proteins (for example, Monomeric Kusabira-Orange, mKOK, mKO2, mOrange, and mOrange2), red fluorescent proteins (for example, mRaspberry, mCherry, mStrawberry, mTangerine, tdTomato, TagRFP, TagRFP-T, mApple, and mRuby), far-red fluorescent proteins (for example, mPlum, HcRed-Tandem, mKate2, mNeptune, and NirFP), near-IR fluorescent proteins (for example, TagRFP657, IFP1.4, and iRFP), long stokes-shift proteins (for example, mKeima Red, LSS-mKate1, and LSS-mKate2), photoactivatible fluorescent proteins (for example, PA-GFP, PAmCherryl, and PATagRFP), photoconvertible fluorescent proteins (for example, Kaede (green), Kaede (red), KikGR1 (green), KikGR1 (red), PS-CFP2, PS-CFP2, mEos2 (green), mEos2 (red), PSmOrange, and PSmOrange), and photoswitchable fluorescent proteins (for example, Dronpa). In an embodiment, the luminescent or fluorescent protein is selected from a green fluorescent protein (GFP), red fluorescent protein (RFP), or mCherry.

In various embodiments, the measurable phenotype is detected or quantified by one or more of dye staining; immunochemistry, gene expression analysis (e.g., qRT-PCR), polynucleotide sequencing, and/or polynucleotide hybridization analysis, such as microarray or FISH.

In various embodiments, the screening involves adding microbial strains, or material derived from cell cultures, to cells or organisms exhibiting a measurable phenotype so as to identify secondary metabolites which can affect the measurable phenotype. In some embodiments, the microbial strain is fed to the cell or organism, for example, where the cell or organism is bacterivorous. In other embodiments, the microbial strain is added to the cell or organism and is engineered to lyse, optionally upon a select stimulation. In such embodiments, the organism may be non-bacterivorous. In various embodiments, the present methods provides the advantage of eliminating the need to extract, concentrate, and/or purify substantial amounts of secondary metabolites for screening thereby enabling a rapid, direct, and parallelized testing of multiple biosynthetic pathways.

In some embodiments, the recipient cells or organisms are plated into wells of a multiwell plate. For example, each well may include about 100 to about 100,000 cells per well, such as from about 1000 to about 10,000 cells per well. When using multicellular organisms, each well may contain from 1 to 100 organisms or from 5 to 100 organisms or from 10 to 50 organisms. In some embodiments, there is at least one well containing control cells or organisms that do not exhibit a measurable phenotype.

In an exemplary embodiments, the present methods provides for the screening of C. elegans having a measurable phenotype. C. elegans are natural bacteriovores that consume bacteria by pharyngeal grinding. In addition, the tissues of C. elegans are transparent at all developmental stages thereby allowing the use of fluorescent probes and tissue-specific fluorescent transgenic markers to study physiological processes in vivo. Developmentally, it takes C. elegans only 3 days to become an adult through four larval stages, L1, L2, L3, and L4, after hatching from its egg. In some embodiments, C. elegans are dispensed into wells at the L1 stage, L2 stage, L3 stage, L4 stage, dauer stage, or adult stage. In some embodiments, the C. elegans are contacted with the microbial strain (or cell culture material) at the L1 stage, L2 stage, L3 stage, L4 stage, dauer stage, or adult stage.

In various embodiments, the C. elegans screening system comprises features described in one more of U.S. Pat. No. 8,809,617, US 2006/0191023, US 2014/0082757, and WO 2000/063424, each of which is hereby incorporated by reference in its entirety. In some embodiments, the C. elegans is engineered to express one or more transgenes including one or more reporter genes, human genes, and/or pharmaceutical targets, or markers for toxicity. In some embodiments, C. elegans is engineered to model one or more aspects of any one of the disease or disorders described herein. For example, the C. elegans may be engineered to model myotonic dystrophy (locomotive disorder), inflammatory bowel disease (inflammatory disorder), lysosomal storage disorders, or cystic fibrosis. In some embodiments, the C. elegans may be engineered to model stress responses, pathogen responses, calcium influx (neuronal signaling), fat storage, developmental timing, brood size, and behavior (e.g., social feeding, food avoidance etc.).

In some embodiments, the microbial strains or material derived from cultures are screened in vitro against purified or isolated molecular targets. In some embodiments, microbial cells are used for screening (either against whole cells or organisms or in vitro assay targets), and the microbial strain is engineered to lyse upon a selected stimulus in order to release its contents including the secondary metabolites. In some embodiments, the strain allows for inducible expression of a protein or enzyme that facilitates lysis, such as lysozyme or a porin, and which may be produced in response to a chemical signal such as the quorum sensing inducer N-3-oxohexanoyl-L-homoserine lactone. See Lorenzo Pasotti, et al., (2011) Characterization of a synthetic bacterial self-destruction device for programmed cell death and for recombinant proteins release. Journal of Biological Engineering. 5:8.

In some embodiments, the microbial strains or cell culture material are plated into wells of a multiwell plate. In some embodiments, each well may include about 100 to 1,000,000 microbial cells per well. For example, each well may include from 1 to about 100 discrete strains for screening, each strain potentially producing a different candidate compound based on a natural product scaffold. For example, each well may include about 1,000, about 5,000, about 10,000, about 100,000, about 500,000, about 1,000,000 microbial cells or more.

In various embodiments, the present methods contemplate the screening of a library of microbial strains each producing a different secondary metabolite based on a selected natural product scaffold. In various embodiments, the library of microbial strains or cell culture material is contacted with the cell, organism, or an in vitro assay target as described herein in separate wells. In some embodiments, the method further comprises identifying a target of an identified candidate molecule. For example, the method may provide for identifying a target of terpenoid molecule identified through screening of a terpenoid library.

In various embodiments, the screening of secondary metabolites is conducted in a high throughput format. In some embodiments, at least about 10 to about 1,000,000 wells, or more, are screened. For example, in various embodiments the present invention provides for screening at least about 100 wells, or at least about 200 wells, or at least about 500 wells, or at least about 1,000 wells, or at least about 2,000 wells, or at least about 5,000 wells, or at least about 10,000 wells, or at least about 50,000 wells, or at least about 100,000 wells, or at least about 500,000 wells, or at least about 1,000,000 wells. In some embodiments, a liquid-based high throughput method is used.

After identification of hits in initial screens, candidate molecules can be diversified for subsequent screens by starting with the corresponding pathway enzymes (from the corresponding microbial cell), and conducting a subsequent round of library generation (e.g. by combinatorial mutagenesis of pathway enzymes). In some embodiments, additional downstream enzymes are added to the pathway, such as library of P450 oxidase enzymes and/or UGT enzymes (or other enzyme described herein), which can be diversified as described above. Final products can be produced by fermentation from microbial cells optimized for high yield production of the identified bioactive metabolite.

In various embodiments, the present methods involve the screening and identification of secondary metabolites for agricultural, industrial, pest control, or therapeutic applications.

In an embodiment, the methods of the invention provides for the screening and identification of secondary metabolites having fungicidal, pesticidal, or anti-parasitic activity. For example, the secondary metabolite may have antihelminthic activity. In such embodiments, the secondary metabolites identified herewith may be formulated as a fungicide or pesticide.

In an embodiment, the methods provide for the screening and identification of secondary metabolites having herbicidal activity, effects on plant growth, or pathogen or pest resistance. In such embodiments, the secondary metabolites identified herewith may be formulated for plant applications.

In an embodiment, the methods provide for the screening and identification of secondary metabolites having insecticidal activity, activity for blocking insect development, or activity as an insect repellant. In such embodiments, the secondary metabolites identified herewith may be formulated as an insecticide or repellant.

In an embodiment, methods provide for the screening and identification of secondary metabolites having pharmaceutical or therapeutic activity. In such embodiments, the secondary metabolites identified herewith may be formulated as a pharmaceutical composition for the treatment of diseases and disorders. Exemplary diseases and disorders that may be treated by the pharmaceutical compositions of the invention include, but are not limited to: cancer; bacterial, viral, or parasitic infection; immune or inflammatory disorders; autoimmune diseases; genetic diseases (including lysosomal storage diseases), cardiovascular diseases; wound healing; ischemia-related diseases, neurodegenerative diseases, metabolic diseases and many other diseases and disorders.

In various embodiments, the pharmaceutical composition may be formulated for any mode of administration as deemed appropriate by a person skilled in the art. Exemplary routes of administration include, for example, oral, dermal, intramuscular, intraperitoneal, intravenous, subcutaneous, intranasal, epidural, sublingual, intratumoral, intracerebral, intravaginal, transdermal, intraocular, rectal, by inhalation, or topical. Administration can be local (e.g., topical) or systemic. In some embodiments, the administering is effected orally. In another embodiment, the administration is by parenteral injection.

Other aspects and embodiments of the invention will be apparent to one or ordinary skill in the art.

### EXAMPLES

### Example 1. Construction of a Library of Terpenoid-Producing E. coli Strains

Identify and Annotate Putative TPSs, P450s and UGTs Through Genome Mining and Transcriptome Analysis from Databases

A library of terpene synthase (TPS) genes was developed from preliminary bioinformatics and literature analysis using two basic criteria. The first was to focus on known terpene biochemical pathways that produce di- or tri-terpenoid molecules with potential drug-relevant scaffolds. The second was to focus on sourcing enzymes from plants and fungi that have previously been reported to have medicinal effects and active terpene metabolism. The library contained 361 plant-derived TPS sequences including 10% putative sesquiterpene synthases, 40% putative diterpene synthases, and 50% putative triterpene synthases. Approximately 100 TPSs were well-characterized and functionally annotated. In addition, 493 putative P450 enzymes and their associated reductase (CPR) enzyme sequences have been identified.

Apply Advanced Bioinformatics Algorithms to Optimize Sequences at the Nucleotide and Amino Acid Levels for Superior Transcriptional, Translational and Folding Efficiency in E. coli.

A bioinformatics workflow was developed to ensure optimal folding, expression, and activity of heterologous enzymes in E. coli. To functionally express plant and fungal TPSs, P450s and UGTs, DNA sequences are designed in silico using algorithms that aim to maximize terpenoid production while minimizing cellular metabolic burden and high translation rates which may lead to misfolded, inactive enzymes. This strategy is in stark contrast to traditional codon optimization algorithms that typically aim to maximize protein expression within a host, generally with the goal of purifying large quantities of the protein.

To accomplish this, the algorithms produce nucleic acid sequences that are indistinguishable from native E. coli genes using a variety of metrics, including mRNA folding energy, the frequency and distribution of anti-Shine-Dalgarno sequences, and codon usage. This design strategy focuses on creating heterologous enzyme sequences that are compatible with the host and hence more likely to form productive pathways for generating the compound of interest. In case DNA coding optimization alone is insufficient to produce functional enzymes, a small panel of protein modifications to the enzyme can be incorporated. These modifications primarily focus on the N-terminus of the protein but also can include globally stabilizing mutations outside of the N-terminus. The N-terminus of TPSs can affect catalysis as it folds back over the C-terminal active site. Likewise, the N-terminus of membrane associated P450 enzymes anchors them to the membrane and can affect substrate uptake. For example, replacing the N-terminal anchor of P450 enzymes with optimized native E. coli membrane tags can enhance P450 oxidation activity and reduce cellular stress. The sequence designs use validated N-terminal modifications that increase the probability that the enzymes display high levels of activity in a heterologous context. By optimizing both the N-terminal sequences and DNA coding using well-developed algorithms and designs, the strategies significantly increase the likelihood of functional expression of heterologous TPS, P450 and UGT enzymes in E. coli.

Design and construct a library of ˜10,000 engineered E. coli strains capable of synthesizing a ˜8,000 molecule terpenoid library through combinatorial pathway assembly and modular metabolic engineering

An initial goal in the application of the technology described herein is to construct a terpenoid library consisting of about 10,000 clones. This is expected to generate 8,000 unique molecules. Screening this library is expected to generate approximately 20-25 hits when used in conjunction with a particular C. elegans disease model to monitor the bioactivity of the terpenoid compounds, which can then be further characterized and validated through genetic analysis and cell-based assays to yield at least ˜5 prioritized lead drug candidates.

To develop the natural product library, a three-step library construction approach using combinatorial pathway assembly and the principles of multivariate modular metabolic engineering (MMME) is undertaken to ensure maximum product diversity.

The first step involves semi-combinatorial MMME pathway optimization of the native E. coli upstream terpenoid pathway and a set of TPS in order to generate an extensive library of sesqui-, di-, and tri-terpene scaffolds. The expression of a single TPS enzyme generally leads to the production of 3-5 terpene scaffolds, the proportions of which can be tuned by varying the relative expressions of the upstream MEP module (enzymes leading to IPP and DMAPP overproduction) and the downstream TPS. To enrich for rare structural isomers, semi-combinatorial libraries of E. coli strains is generated in which the expression of each of these modules is simultaneously modulated through the use of three promoters spanning a range of strengths. By evaluating an initial set of 1000 TPS enzymes (3 MEP module promoters by 3 TPS promoters for 1000 TPS enzymes leading to a total of 9000 strains), it is expected that between 3000-5000 unique terpene scaffolds are obtained. Strains carrying these putative terpene pathways are cultured and analyzed by GC-MS to determine the function and product profiles contained within this library. This analysis will lead to the selection of at least 2000 strains, each of which will be enriched in the production of a unique terpene scaffold, for further combinatorial engineering.

The second step focuses on adding and tuning P450 pathway expression to synthesize oxygenated products from these terpene scaffolds. Approximately 300 P450 genes are synthesized with specific N-terminal tags for functional expression in E. coli. For each selected strain from the first step, a P450 pathway (comprised of two P450 enzymes and one cytochrome P450 reductase (CPR) gene expressed as an operon) is expressed using three different promoter strengths in order to generate a panel of oxygenated terpenoid products. The P450 enzymes paired with each TPS are selected by bioinformatics analysis to increase the likelihood of reactivity with the appropriate terpene substrate or, alternatively, are determined by specific terpenoid pathways which have been characterized for known molecules, such as those derived from fungal genome mining. From this set of 6000 strains (2000 terpene scaffold strains expressing a P450 pathway under three different promoters), it is expected that 4000 unique oxygenated terpene molecules are obtained based on past studies which have shown an average production of 3-5 oxygenated molecules generated from single P450 enzymes acting on taxadiene, kaurene, and various sesquiterpene scaffolds. Strains carrying these putative terpene pathways are cultured and analyzed by GC-MS to determine the function and product profiles contained within this library.

In the third step, UGT enzymes are incorporated to increase the diversity of the oxygenated terpenoids through glycosylation. To further diversify and improve the functionality of the oxygenated terpene scaffolds, a set of 500 strains is selected from the second step which expresses high levels of a broad range of oxygenated terpenoids, and UGT enzymes are incorporated to mediate O-glucosylation of the scaffolds. During this step, approximately 200 UGT genes (both native and designed using a proprietary domain shuffling technology) are synthesized and used to create a library by expressing two UGTs as operons under three different expression systems in the background of the prioritized strains from step two (500 oxygenated terpenoid strains by 3 UGT module promoters leading to 1500 strains). It is expected that 2000 glycosylated scaffolds (˜4 glycosylated molecules per starting clone) are generated.

Altogether, the 2000 terpene scaffold strains selected in step one, 6000 strains constructed in step two, and 1500 strains constructed in step three (with an estimated total compound library size of 8000 distinct molecules) are screened for bioactivity and toxicity using the C. elegans assay described below.

Analytical methods (GC-MS or LC-MS) are utilized at each step to characterize the product profiles of engineered strains and to prioritize the sets of strains being carried forward at certain stages to ensure sufficient compound diversity in our library.

Using the technology platform described above, and by mining the genomes and transcriptomes of six different citrus plant species from public databases, 64 putative TPS enzymes have been identified. These novel, previously uncharacterized citrus genes were then used to construct 384 E. coli strains (2 upstream MEP modules variants and 3 promoter strengths per TPS) using the MMME pathway engineering technology. GC-MS analysis identified a total of 68 distinct terpenoid molecules (mostly sesquiterpenoids), with each analyzed strain producing 3-10 different terpenoid molecules. As demonstrated in prior studies, pathway modulation was successful in enriching for several rare molecules. This library was used for the C. elegans screening platform studies described below.

### Example 2. High Throughput Screening of Terpenoid-Producing E. coli Strains in C. elegans Disease Models

The overall goals of this example is to screen 10,000 terpenoid-producing E. coli strains generated as previously described in two C. elegans disease models and to validate and prioritize the hits by genetic analysis and additional cell-based assays. A further goal is to use previously developed metabolic and bioprocess engineering technologies to generate and ferment E. coli strains that produce high yields of prioritized compounds for structural analysis and for efficacy testing in appropriate cell models.

Screen the Library of E. coli Strains Generated in Example 1 in a Variety of Whole-Animal C. elegans Assays, Including Disease Models for IBD and DM1

Initially, terpenoid-producing strains are tested for their ability to cause developmental arrest in order to ascertain the level of toxicity of the produced compounds. Briefly, 15 L1 stage animals are added to each well of a 384 well plate containing control or terpenoid-producing E. coli strains using the COPAS biosorter. After incubation at 25° C. for 72 hours, the worms are imaged using a IXMicro automated microscope. Automated CellProfiler analysis is used to identify any animals that are smaller than expected and to determine any developmental stage of arrest and/or morphological defects. Preliminary studies utilizing a citrus-derived natural product library of E. coli clones demonstrated that none of the terpenoids trigger non-specific developmental, stress, immune or lifespan defects in C. elegans. In addition, transcription profiling of 20 representative stress and immune-related genes and 74 intestine-related genes revealed no terpenoid-activated gene expression, even with pgp-1 or pgp-3 export pump mutants, which enable increased uptake of environmental molecules. These results indicate that terpenoids in general are not toxic, do not activate detoxification enzymes, and do not function as “promiscuous” hitters in various assays in C. elegans.

Terpenoid-producing strains are also tested for their ability to activate/repress the expression of a variety of GFP reporter genes corresponding to conserved signal transduction pathways. The goal is to test the prediction that terpenoids exhibit a broad range of bioactivity in activating or suppressing evolutionarily-conserved signal transduction pathways. The pathways to be tested include the p38 MAPK signaling pathway involved in innate immune signaling, the Wnt signaling pathway involved in both development and immune signaling, the insulin signaling pathway, pathways that respond to reactive oxygen species, osmotic stress, heat shock, and genes involved in the unfolded protein response.

To demonstrate the potential of this drug discovery platform, two C. elegans disease models are used. The first relates to diseases of the intestinal epithelium such as inflammatory bowel disease (IBD). It is expected that this screen may identify compounds that could activate or repress key components of the innate immune response or function as agonists or antagonists of immune-associated receptors such as G protein-coupled receptors (GPCRs). The second C. elegans disease model relates to RNA toxicity diseases, such as myotonic dystrophy type 1 (DM1), which is caused by mRNA aggregation of the dystrophia myotonica-protein kinase (DMPK) gene. It is expected that this screen can identify compounds that act directly on aberrant RNAs, on RNA clearance pathways such as nonsense-mediated mRNA decay, or on enhancing muscle repair.

Specifically, a screen is carried out to identify terpenoid-producing strains that activate innate immune responses in a C. elegans IBD model. Specifically, the Sytox worm survival assay is used to identify terpenoids that block the ability of E. faecalis to kill nematodes. This screen is expected to identify terpenoids that can 1) enhance host immunity, allowing C. elegans to rapidly kill an invading pathogen, 2) enhance host resilience thereby allowing C. elegans to survive tissue damage (inflammation) that would otherwise kill it, 3) kill the pathogen, or 4) block the virulence of the pathogen. As a secondary assay, hits are tested in a C. elegans infection assay in which GFP-expressing E. faecalis is counter-screened with propidium iodide (red), which only stains dead bacteria. The red/green ratio serves as a proxy for the effectiveness of the immune response and distinguishes between compounds that kill the pathogen, enhance immunity, or affect the ability of the pathogen to colonize the intestine.

Additionally, a screen is carried out to identify terpenoid-producing strains for their ability to block RNA toxicity. Specifically, terpenoids are identified which rescue the loss of motility or reduced GFP fluorescence exhibited by the C. elegans DM1 model, in which the nematodes express expanded RNA repeats (e.g., 123 CUG repeats) in the 3′ untranslated region of the GFP gene expressed in body wall muscle cells and in which the nematodes exhibit phenotypes also observed in myotonic dystrophy patients including reduced muscle strength and the formation of nuclear foci that interact with the C. elegans ortholog of the human MBNL1 (muscle-blind) protein. Garcia, S. M., et al., Identification of genes in toxicity pathways of trinucleotide-repeat RNA in C. elegans. Nat. Struct. Mol. Biol. 21:712-720 (2014). In this screen, the ability of the terpenoid-producing E. coli strains to restore GFP expression in the DM1 animals is screened using an automated fluorescent microscope, or the ability of the terpenoid-producing E. coli strains to improve the motility of DM1 animals is screened using WMicroTracker, a commercial instrument for monitoring C. elegans movement.

Additional screens are carried out using C. elegans disease models of, for example, lysosome diseases, cystic fibrosis, and diseases associated with protein folding defects.

### Use Genetic Tools and Next Generation Sequence (NGS) Analysis to Identify the Putative Gene Targets of a Selected Subset of Lead Active Compounds

Genetic and NGS analysis are used to identify the putative gene targets of the hits identified from the various screens. It is expected that the hit compounds target genes and pathways known to be relevant to IBD and RNA toxicity.

About 20-25 hits in each of the IBD and DM1 screens are expected to be generated by screening a 10,000 member E. coli terpenoid library. The hits are prioritized on the basis of rescue strength, toxicity, and off target effects. The molecular target(s) of ˜10 selected prioritized terpenoids are identified through mutagenesis screening and whole-genome sequencing. In the case of DM1-related hits, suppressor mutants are identified by feeding EMS-mutagenized worms the terpenoid-expressing E. coli and screening for animals that now retain their RNA toxicity phenotype. NGS is used to identify the gene(s) mutated in these suppressors, and results are subsequently confirmed through rescue experiments.

Hits that target highly predicted pathways (e.g. GPCRs and p38 MAPK signaling for IBD and RNA splicing, alternative splicing factors, RNA clearance, and RNA interactions for DM1) are prioritized for further analysis.

Produce and Purify High Levels of Prioritized Selected Lead Compounds in E. coli, Identify the Molecular Structure of Compounds with Promising Bioactivity, and Validate the Compounds in Mammalian Tissue Culture Models

Prioritized terpenoid-producing E. coli strains are used to generate large quantities of pure terpenoid lead compounds. Strains producing ˜50-100 mg/L of terpenoid mixtures in a 96 deep well plate assay can be scaled up to >2 g/L. These molecules are extracted and purified using established facilities for downstream purification (e.g. FLASH chromatography with an ELSD detector which enables purification up to grams of material). Pure compounds are reconfirmed for bioactivity using C. elegans assays, which require <10 mg of compound. The structures of promising compounds are elucidated through NMR characterization. Purified terpenoids are further characterized and prioritized using cell-based assays for IBD and DM1. For example, IBD lead compounds are tested in a Salmonella enterica intracellular replication assay in HeLa cells, a validated immune-competence assay, to determine whether immune-related genes identified in C. elegans also play a role in the human immune response. Similarly, DM1 lead compounds are tested for their effects on RNA foci accumulation in human fibroblast DM1 models (one of the hallmarks of RNA toxicity disorders) and on reverting alternative splicing defects, which are known to be disrupted in DM1.

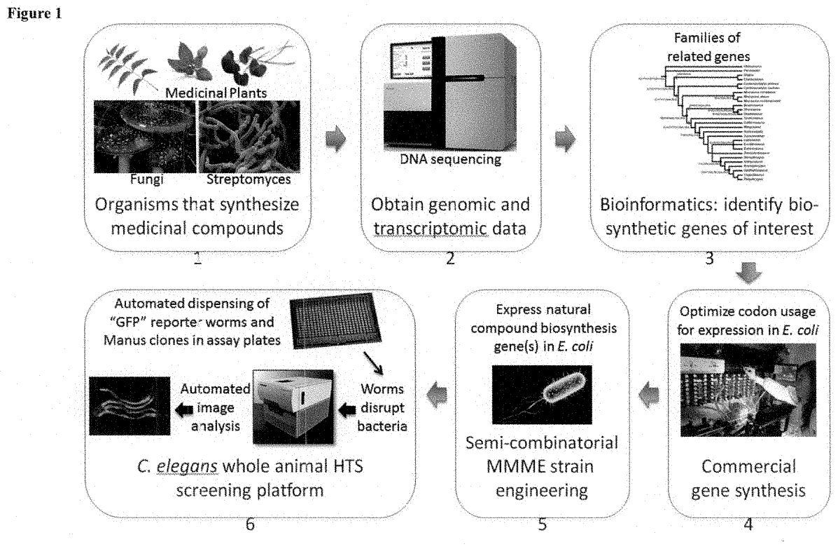

An exemplary schematic of E. coli library construction and high throughput screening of the library using C. elegans disease models is provided in FIG. 1.

### Example 3. Screening for Secondary Metabolites Producing a Measurable Phenotype in C. elegans

Terpenoids secreted by engineered bacteria were collected in an oil overlay which was then fed to C. elegans animals from their first larval stage through adulthood. The effect of the terpenoid(s) on C. elegans was determined by qRT-PCR analysis measuring C. elegans gene expression (Table 2). This analysis included genes generally related to C. elegans stress and immune responses. Ingesting specific terpenoids resulted in upregulation of some C. elegans genes as compared to control conditions (shown in bold and italicized font in Table 2). For example, exposure to the sample 34 containing the terpenoids nerolidol (major product) and farnesol (minor product) caused an 18 fold upregulation of the C. elegans spp-9 gene. spp-9 presumptively encodes a saposin like its human homolog PSAP, which is involved in a number of biological functions including breakdown of sphingolipids, and which plays a role in transporting lipids to the outer surface of the cell so that they can be recognized by the immune system. The spp-9 gene was not unregulated in animals exposed to other oil overlays containing different terpenoids indicating that the spp-9 induction was a specific response to the specific terpenoids present in sample 34.

TABLE 2

| Sample | Fold change of the indicated C. elegans gene relative to control conditions | | | | | | | | | | |
|---|---|---|---|---|---|---|---|---|---|---|---|
| number | clec-67 | pgp-6 | irg-1 | arrd-3 | F55G11.2 | spp-9 | pgp-9 | cnc-19 | abf-1 | ily-5 | F08G5.6 |
| 1 | 2.33 | 3.17 | 2.14 | 5.49 | 2.19 | 0.22 | 4.11 | 2.56 | 1.56 | 0.79 | 1.89 |
| 2 | 1.71 | 2.04 | 1.13 | 2.54 | 3.19 | 0.44 | 1.05 | 1.68 | 1.36 | 0.77 | 1.17 |
| 3 | 3.01 | 3.38 | 2.95 | 4.29 | 0.25 | 0.25 | 4.48 | 2.99 | 2.79 | 1.00 | 2.29 |
| 4 | 1.65 | 2.36 | 1.01 | 2.56 | 3.09 | 0.76 | 0.53 | 1.40 | 1.00 | 0.49 | 0.75 |
| 5 | 2.23 | 3.73 | 2.49 | 4.03 | 0.00 | 3.01 | 1.77 | 3.08 | 2.21 | 0.91 | 0.03 |
| 6 | 0.18 | 0.53 | 0.88 | 0.57 | 1.33 | 0.36 | 0.07 | 0.26 | 0.42 | 0.36 | [symbol] |
| 7 | 0.30 | 0.68 | 0.46 | 0.38 | 0.36 | 0.13 | 0.19 | 0.24 | 0.20 | 0.14 | 0.20 |
| 8 | 0.43 | 0.62 | 0.48 | 0.49 | 0.47 | 0.30 | 0.23 | 0.26 | 0.21 | 0.09 | 0.33 |
| 9 | 0.23 | 0.50 | 0.35 | 0.21 | 0.19 | 2.01 | 0.20 | 0.18 | 0.32 | 0.07 | 0.25 |
| 11 | 0.35 | 0.69 | 0.39 | 0.57 | 0.35 | 0.61 | 0.41 | 0.29 | 0.21 | 0.31 | 0.24 |
| 12 | 0.42 | 0.55 | 0.39 | 0.54 | 0.41 | 0.26 | 0.13 | 0.18 | 0.22 | 0.09 | 0.27 |
| 13 | 3.70 | 4.27 | 2.67 | 6.10 | 7.17 | 0.33 | 0.73 | 3.90 | 1.78 | 1.18 | 4.64 |
| 14 | 3.07 | 3.10 | 2.27 | 4.54 | 3.82 | 0.26 | 0.98 | 3.18 | 2.29 | 0.87 | 2.71 |
| 15 | 1.62 | 2.13 | 1.30 | 2.93 | 2.17 | 2.03 | 0.72 | 1.69 | 0.71 | 0.56 | 1.67 |
| 16 | 2.44 | 3.26 | 2.17 | 2.73 | 1.82 | 0.99 | 0.57 | 2.69 | 0.80 | 0.67 | 1.72 |
| 17 | 2.71 | 3.48 | 1.83 | 3.03 | 4.05 | 10.34 | 1.33 | 2.88 | 0.60 | 0.84 | 1.62 |
| 18 | 2.19 | 2.57 | 1.78 | 3.41 | 2.66 | 3.97 | 0.53 | 2.05 | 0.68 | 0.69 | 1.52 |
| 19 | 0.26 | 0.55 | 0.39 | 0.12 | 0.17 | 0.46 | 0.26 | 0.22 | 0.27 | 0.04 | 0.17 |
| 20 | 0.20 | 0.54 | 0.31 | 0.22 | 0.08 | 0.16 | 0.20 | 0.21 | 0.27 | 0.08 | 0.12 |
| 21 | 0.19 | 0.52 | 0.41 | 0.26 | 0.10 | 11.80 | 0.09 | 0.20 | 0.16 | 0.07 | 0.15 |
| 22 | 0.16 | 0.32 | 0.32 | 0.06 | 0.14 | 1.63 | 0.07 | 0.11 | 0.10 | 0.03 | 0.10 |
| 23 | 1.34 | 2.38 | 1.55 | 2.68 | 0.92 | 0.40 | 4.73 | 2.00 | 0.65 | 0.44 | 1.28 |
| 24 | 2.58 | 3.15 | 2.24 | 4.57 | 1.78 | 0.55 | 3.15 | 2.77 | 3.21 | 1.07 | 2.35 |
| 25 | 1.47 | 2.11 | 1.08 | 2.50 | 0.75 | 0.73 | 1.01 | 1.35 | 0.64 | 0.51 | 1.09 |
| 26 | 1.93 | 2.62 | 1.43 | 1.51 | 1.39 | 1.17 | 1.24 | 1.39 | 0.85 | 0.46 | 1.07 |
| 27 | 1.98 | 2.37 | 1.38 | 2.89 | 2.38 | 0.45 | 2.24 | 1.59 | 0.73 | 0.79 | 1.40 |
| 28 | 2.78 | 3.30 | 1.51 | 3.68 | 2.02 | 2.45 | 0.94 | 1.96 | 0.85 | 0.76 | 1.26 |
| 29 | 2.30 | 3.26 | 1.54 | 3.07 | 2.84 | 0.52 | 1.40 | 1.69 | 1.02 | 0.60 | 1.15 |
| 30 | 1.18 | 2.21 | 1.00 | 2.25 | 1.14 | 2.58 | 0.46 | 1.05 | 0.72 | 0.95 | 0.67 |
| 31 | 1.70 | 1.38 | 1.05 | 1.43 | 3.27 | [symbol] | 1.34 | 0.83 | 0.34 | 0.38 | 0.87 |
| 32 | 2.78 | 2.17 | 1.23 | 2.25 | 5.91 | 2.77 | 0.60 | 1.22 | 0.69 | 0.55 | 1.29 |
| 33 | 3.08 | 2.68 | 1.62 | 2.92 | 5.87 | 0.57 | 0.15 | 1.61 | 1.03 | 0.91 | 2.22 |
| 34 | 2.35 | 2.01 | 0.00 | 2.39 | 4.77 | [symbol] | 3.27 | 2.13 | 1.46 | 0.73 | 2.10 |
| 35 | 0.55 | 2.28 | 3.88 | 2.50 | [symbol] | 0.41 | 4.33 | 2.08 | 3.28 | 2.56 | 2.76 |
| 36 | 3.11 | 1.42 | 1.06 | 2.94 | 2.52 | 0.87 | 1.98 | 0.98 | 0.53 | 0.48 | 0.70 |
| 37 | 1.26 | 2.08 | 1.49 | 1.32 | 4.67 | [symbol] | 0.30 | 1.42 | 0.63 | 0.99 | 1.10 |
| 38 | 1.28 | 3.35 | 1.82 | 2.69 | 1.96 | 5.31 | 0.83 | 1.61 | 1.06 | 0.43 | 0.90 |
| 39 | 2.06 | 1.28 | 0.76 | 1.10 | 1.12 | 0.79 | 0.22 | 0.91 | 0.43 | 0.18 | 0.57 |
| 40 | 3.49 | 1.46 | 1.03 | 1.32 | 4.45 | 3.07 | 0.55 | 3.89 | 1.31 | 0.63 | 0.80 |
| 41 | 0.99 | 3.35 | 2.29 | 4.68 | 6.94 | 0.14 | 0.71 | 0.67 | 2.95 | 1.45 | 2.78 |
| 42 | 1.98 | 2.89 | 1.31 | 2.60 | 2.58 | 0.57 | 0.93 | 1.63 | 0.51 | 0.54 | 1.18 |
| 43 | | 1.80 | 1.14 | 1.93 | 1.38 | 0.29 | 5.94 | 1.07 | 0.41 | 0.24 | 0.70 |

1. A method for screening for bioactive agents, comprising:
providing a cell or organism exhibiting a measurable phenotype, wherein the organism is selected from a fungus, protist, hydrozoa, planaria, nematode, insect, plant or plant part, or microbe;
contacting the cell or organism with a microbial library or material derived therefrom, the microbial library producing a library of secondary metabolites through combinatorial expression of synthetic genes, and
identifying secondary metabolites that affect the measurable phenotype.

2. The method of claim 1, wherein microbial strains in the library are fed to an organism exhibiting a measurable phenotype, wherein the organism is optionally bacterivorous.

3. The method of claim 1, wherein the microbial strain is engineered to lyse upon a selected stimulus, and the cell or organism is optionally a cell line or non-bacterivorous organism.

4. The method of claim 1, wherein the organism is a protozoan.

5. The method of claim 1, wherein the organism is a cnidarian.

6. The method of claim 1, wherein the organism is a flatworm.

7. The method of claim 1, wherein the organism is an arthropod.

8. The method of claim 1, wherein the organism is an amoeba or paramecium.

9. The method of claim 1 or 2, wherein the organism is a nematode, which is optionally Caenorhabditis elegans (C. elegans).

10. The method of claim 3, wherein the organism or cell is a plant cell, plant, or plant part.

11. The method of claim 3, wherein the organism or cell is a cell of a vertebrate organism, or an embryo.

12. The method of claim 3, wherein the cell or organism is a fungi or yeast.

13. The method of any one of claims 1 to 12, wherein the organisms or cells are plated in wells of a multiwell plate.

14. The method of claim 13, wherein from 5 to 100 organisms are deposited per well; or from 100 to 100,000 cells are deposited per well.

15. The method of claim 13 or 14, wherein at least one well contains control cells or organisms that do not exhibit the measurable phenotype.

16. The method of any one of claims 13 to 15, wherein at least about 100 wells are screened.

17. The method of any one of claims 13 to 16, wherein the organism is C. elegans, and worms are dispensed into wells at L1 stage, L2 stage, L3 stage, L4 stage, dauer stage, or adult stage.

18. The method of any one of claims 13 to 16, wherein the organism is C. elegans, and the C. elegans are contacted with the microbial strain or material derived therefrom at L1 stage, L2 stage, L3 stage, L4 stage, dauer stage, or adult stage.

19. The method of claim 17 or 18, wherein the C. elegans are screened in high throughput.

20. The method of any one of claims 1 to 19, wherein the effect on said measurable phenotype is quantified by the level of protein expression of a reporter gene and/or cellular location of the reporter gene RNA or protein, or impact on morphology or motility.

21. The method of claim 20, wherein the reporter gene is a fluorescent or luminescent protein.

22. The method of claim 21, wherein the agent increases reporter gene detection.

23. The method of claim 22, wherein the agent decreases reporter gene detection.

24. The method of any one of claims 1 to 23, wherein the microbial strain is a bacterium.

25. The method of claim 24, wherein the bacterium is E. coli, Pseudomonas spp., Enterococcus spp., Bacillus spp., or Staphylococcus spp.

26. The method of any one of claims 1 to 23, wherein the microbial strain is an archaea.

27. The method of any one of claims 1 to 23, wherein the microbial strain is a fungus or yeast.

28. The method of any one of claims 1 to 27, wherein the cell or organism is contacted with secondary metabolite recovered from cultures in an organic or hydrophobic phase.

29. The method of any one of claims 1 to 28, wherein the measurable phenotype is detected or quantified by: dye staining; immunochemistry, gene expression analysis, which is optionally by qRT-PCR, polynucleotide sequencing, and/or polynucleotide hybridization analysis, such as microarray or FISH.

30. The method of any one of claims 1 to 29, wherein the cell or organism expresses a human gene.

31. The method of any one of claims 1 to 30, wherein the measurable phenotype is induction or reduction of gene expression or protein expression, protein modification, metabolism, change in metabolic or physiologic state, subcellular or tissue structure and organization, protein or RNA stability, epigenetic modification, cell or organism death, lifespan extension, autophagy, organellar structure and function, intracellular or intercellular trafficking or signaling, neuronal functioning, cell proliferation, RNA toxicity, a stress response, a pathogen response, calcium influx, fat storage, developmental timing, brood size, or behavior such as social feeding or food avoidance.

32. The method of any one of claims 1 to 30, wherein the measurable phenotype is determined by assaying pathogen response, stress response, detoxification, hypoxia response, unfolded protein response, mitochondrial marker(s), RNAi function, piRNA function, microRNA function, proteasome function, and/or the measurable phenotype is the activity of a subcellular or intercellular signaling pathway.

33. An in vitro method for screening for active agents, comprising: providing a microbial strain that has been engineered to lyse upon a selected stimulus and which produces a library of secondary metabolites synthesized by combinatorial expression of one or more heterologous genes, adding the microbial strain or material derived therefrom to an in vitro assay, and identifying whether the secondary metabolite has a measurable activity in the in vitro assay.

34. The method of claim 33, wherein the microbial strain or material derived therefrom is plated in wells of a multiwell plate.

35. The method of claim 33 or 34, wherein al least about 100 wells are screened.

36. The method of any one of claims 33 to 35, wherein the microbial strain is a bacterium.

37. The method of claim 36, wherein the bacterium is E. coli, Pseudomonas spp., Enterococcus spp., Bacillus spp., and Staphylococcus spp.

38. The method of any one of claims 33 to 35, wherein the microbial strain is an archaea.

39. The method of any one of claims 33 to 35, wherein the microbial strain is a fungus or yeast.

40. The method of any one of claims 1 to 39, wherein the secondary metabolite is a terpene, terpenoid, alkaloid, cannabinoid, steroid, saponin, glycoside, stilbenoid, polyphenol, flavonoid, antibiotic, polyketide, fatty acid, or a non-ribosomal peptide.

41. The method of claim 40, wherein the secondary metabolite is a terpene or terpenoid.

42. The method of claim 41, wherein the secondary metabolite is a monoterpene or monoterpenoid, a sesquiterpene or sesquiterpenoid, a diterpene or diterpenoid, sesterterpene or sesterterpenoid, or a triterpene or triterpenoid.

43. The method of any one of claims 1 to 42, wherein the library of microbial strains expresses a library of terpene synthases.

44. The method of claim 43, wherein the microbial strain is an E. coli that expresses one or more additional copies of an MEP pathway enzyme, which is optionally one or more of dxs, ispD, ispF, and/or idi genes.

45. The method of claim 43 or 44, wherein the microbial strain overexpresses one or more of a geranyl diphosphate synthase (GPS), a geranylgeranyl diphosphate synthase (GGPS), a farnesyl diphosphate synthase (FPS), and a farnesyl geranyl diphosphate synthase (FGPPS).

46. The method of any one of claims 40 to 45, wherein the microbial strains express a library of terpenoid synthase enzymes.

47. The method of any one of claims 41 to 46, wherein the microbial strains express a library of P450 oxidase enzymes.

48. The method of any one of claims 41 to 47, wherein the microbial strains express a library of uridine diphosphate dependent glycosyltransferase (UGT) enzymes, methyltransferase enzymes, acetyltransferase enzymes, and/or benzoyl transferase enzymes.

49. The method of any one of claims 44 to 47, wherein the microbial library expresses pathway enzymes in at least two modules, with expression levels of the modules varied in the library by at least two or at least three promoter strengths.

50. The method of any one of claims 1 to 49, wherein a library of microbial strains or material derived therefrom, each producing a different secondary metabolite, are contacted with the cell, organism, or in vitro assay target in separate wells.

51. The method of claim 50, further comprising, identifying the target of the identified secondary metabolite.

52. The method of any one of claims 1 to 51, wherein bioactive secondary metabolites are produced by fermentation of corresponding microbial strains, optionally optimized for production yield.

53. The method of any one of claims 1 to 52, wherein the cell or organism is a fungus, nematode, or protozoan, and the secondary metabolites are screened for fungicidal, pesticidal, or anti-parasitic activity, which is optionally antihelminthic.

54. The method of claim 53, further comprising formulating the identified agent as a fungicide or pesticide.

55. The method of any one of claims 1 to 52, wherein the cell or organism is a plant or plant cell, and the secondary metabolites are screened for herbicidal activity, effect on plant growth, or pathogen or pest resistance.

56. The method of claim 55, further comprising formulating the identified agent for application to plants.

57. The method of any one of claims 1 to 52, wherein the cell or organism is an insect or insect cell or embryo, and the secondary metabolites are screened for insecticidal activity, activity for blocking development, or activity as a repellant.

58. The method of claim 57, wherein the identified agent is formulated as an insecticide or repellant.

59. The method of any one of claims 1 to 52, wherein the secondary metabolites are screened for pharmaceutical activity.

60. The method of claim 59, further comprising formulating the identified agent as a pharmaceutical composition.

61. The method of claim 60, wherein the agent is formulated for systemic administration, which is optionally by the oral route.
